(19) United States
(12) Patent Application Publication
(10) Pub. No.: US 2023/0211071 A1
(43) Pub. Date: Jul. 6, 2023

(54) ANCHORABLE MEDICAL FLUID EXTENSION SET SYSTEM

(71) Applicant: CareFusion 303, Inc., San Diego, CA (US)

(72) Inventors: Jason Andrew Wine, Brea, CA (US); Ryan Callahan, Long Beach, CA (US); Jon K. Burkholz, Salt Lake City, UT (US); Tomas Frausto, Walnut, CA (US); Siddarth Shevgoor, Mission Viejo, CA (US)

(21) Appl. No.: 18/081,258

(22) Filed: Dec. 14, 2022

Related U.S. Application Data
(60) Provisional application No. 63/295,346, filed on Dec. 30, 2021.

(51) Int. Cl. A61M 5/14; A61M 25/02

(52) U.S. Cl. CPC A61M 5/1411; A61M 25/02; A61M 2025/0266

(57) ABSTRACT

Medical fluid extension set systems are disclosed that can include a tubing anchor coupled to tubing and a fluid coupling device, and can be anchored to a patient to resist unintended separation of the tubing anchor or a catheter coupled to the tubing, from a patient, and to permit separation or disconnection of a fluid pathway between the tubing and another device coupled to thereto upon exceeding a pulling force on the fluid coupling device, and where disconnection includes separation of first and second valve assemblies such that a fluid pathway of the first and second valve assemblies is obstructed upon disconnection of the fluid coupling device.

### CROSS-REFERENCE TO RELATED APPLICATIONS

This application claims the benefit of U.S. Provisional Application No. 63/295,346, filed Dec. 30, 2021, the entirety of which is incorporated herein by reference.

### BACKGROUND

The present disclosure relates generally to medical fluid connectors and, more particularly, to anchorable medical fluid extension set systems that can be affixed to a patient to resist unintended separation of a medical fluid tubing, such as a catheter, from a patient, and which can include a medical connection.

Medical connections are widely used in fluid delivery systems such as those used in connection with intravenous (IV) fluid lines or tubing, blood access, hemodialysis, peritoneal dialysis, enteral feeding, drug vial access, and other procedures. Tubing can be used for a variety of therapies that may require accessing an internal portion of a patient, including, for example IV therapy and enteral feeding. An internal portion of a patient can be accessed by creating a pathway that penetrates or enters into the patient through a natural or artificial opening of the patient. The pathway can be created or maintained using a device, such as an IV set including a catheter coupled to tubing.

In some instances, a tubing for medical fluid, such as an IV set, can be unintentionally dislodged or separated from the patient when an unintended force is exerted on the tubing, the catheter, and/or another portion of an IV set or fluid pathway connected to a patient. For example, an IV set can be subjected to an unintended force when the patient moves or rolls over within a bed, or when the tubing or another portion of an IV set become caught on a portion of the bed, such as the railing, or when a patient is panicking, disoriented, or fidgeting such that the IV set is unintentionally or intentionally pulled away from the patient or away from the medical equipment coupled to the tubing.

### SUMMARY

In accordance with at least some embodiments disclosed herein is the realization that unintended dislodgement or disconnection of a medical connection, such as a medical fluid line, can result in injury to a patient or a caregiver, such as by depriving the patient of a medicament, increasing the potential for infection to the patient, and exposing the caregiver to harmful medicaments.

Aspects of the present disclosure provide methods of providing a medical fluid extension set system, the method comprising providing a tubing having a first end and a second end, wherein the first end comprises a fluid coupling device; and coupling a tubing anchor to the tubing, such that the tubing anchor is positioned between the first and second ends of the tubing.

Accordingly, the present application addresses several operational challenges encountered in prior fluid connections and provides numerous improvements that enable the user to increase safety and efficacy, while more easily and precisely providing fluid connections.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a medical fluid extension set system intravenously coupled to a patient, in accordance with aspects of the present disclosure.

FIG. 2 illustrates the medical fluid extension set system of FIG. 1 being pulled in a direction away from the patient, in accordance with aspects of the present disclosure.

FIG. 3 illustrates reassembly of a portion of the medical fluid extension set system of FIG. 1, in accordance with aspects of the present disclosure.

FIG. 4 illustrates a tubing anchor in use with an IV set intravenously coupled to a patient, in accordance with aspects of the present disclosure.

FIG. 5 illustrates a tubing anchor coupled to portion of a medical fluid extension set, in accordance with aspects of the present disclosure.

FIG. 6 illustrates a front perspective view of a tubing anchor, in accordance with aspects of the present disclosure.

FIG. 7 illustrates a top elevation view of a tubing anchor, in accordance with aspects of the present disclosure.

FIGS. 8 and 9 illustrate rear perspective views of a tubing anchor, in accordance with aspects of the present disclosure.

FIG. 10 illustrates another embodiment of a medical fluid extension set system, in accordance with aspects of the present disclosure.

FIG. 11 illustrates another embodiment of a medical fluid extension set system, in accordance with aspects of the present disclosure.

FIG. 12 illustrates another embodiment of a medical fluid extension set system, in accordance with aspects of the present disclosure.

FIG. 13 illustrates another embodiment of a medical fluid extension set system, in accordance with aspects of the present disclosure.

FIG. 14 illustrates another embodiment of a medical fluid extension set system, in accordance with aspects of the present disclosure.

FIG. 15 illustrates another embodiment of a medical fluid extension set system, in accordance with aspects of the present disclosure.

FIG. 16 illustrates another embodiment of a medical fluid extension set system, in accordance with aspects of the present disclosure.

FIG. 17 illustrates another embodiment of a medical fluid extension set system, in accordance with aspects of the present disclosure.

FIG. 18 illustrates a perspective view of a fluid coupling device, in accordance with aspects of the present disclosure.

FIGS. 19A and 19B illustrate perspective views of the fluid coupling device of FIG. 18, in accordance with aspects of the present disclosure.

FIG. 20 illustrates a cross-sectional view of the fluid coupling device of FIGS. 19A and 19B, in accordance with aspects of the present disclosure.

FIG. 21 illustrates a cross-sectional view of the fluid coupling device of FIG. 18, in accordance with aspects of the present disclosure.

FIG. 22 illustrates a cross-sectional view of another embodiment of a fluid coupling device, in accordance with aspects of the present disclosure.

FIG. 23 illustrates a perspective view of another embodiment of a fluid coupling device, in accordance with aspects of the present disclosure.

FIGS. 24A and 24B illustrate perspective views of the fluid coupling device of FIG. 23, in accordance with aspects of the present disclosure.

FIG. 25 illustrates a cross-sectional view of the fluid coupling device of FIGS. 24A and 24B, in accordance with aspects of the present disclosure.

FIG. 26 illustrates a cross-sectional view of the fluid coupling device of FIG. 23, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present application discloses various features and advantages of a medical fluid extension set system. The medical fluid extension set system can provide for efficient and safe maintenance of fluid connections, such as the connections used for transferring medical fluids toward or away from a patient. The medical fluid extension set system can maintain a fluid pathway by resisting unintended disconnection when a pulling or tension force is applied to a portion of the medical fluid extension set system, such as when a patient moves or when the medical tubing is pulled away from the patient.

The medical fluid extension set system can also prevent injury to a patient or a caregiver by resisting disconnection or separation of a catheter coupled to the medical fluid extension set system when a pulling or tension force exceeds a threshold. The medical fluid extension set system can also prevent injury to a patient or a caregiver by obstructing the fluid pathway when disconnection or separation between portions of the medical fluid extension set system occurs. Further, the medical fluid extension set system can provide for efficient and safe reestablishment of the fluid pathway, by permitting reassembly of portions of the system after a disconnection or separation occurs.

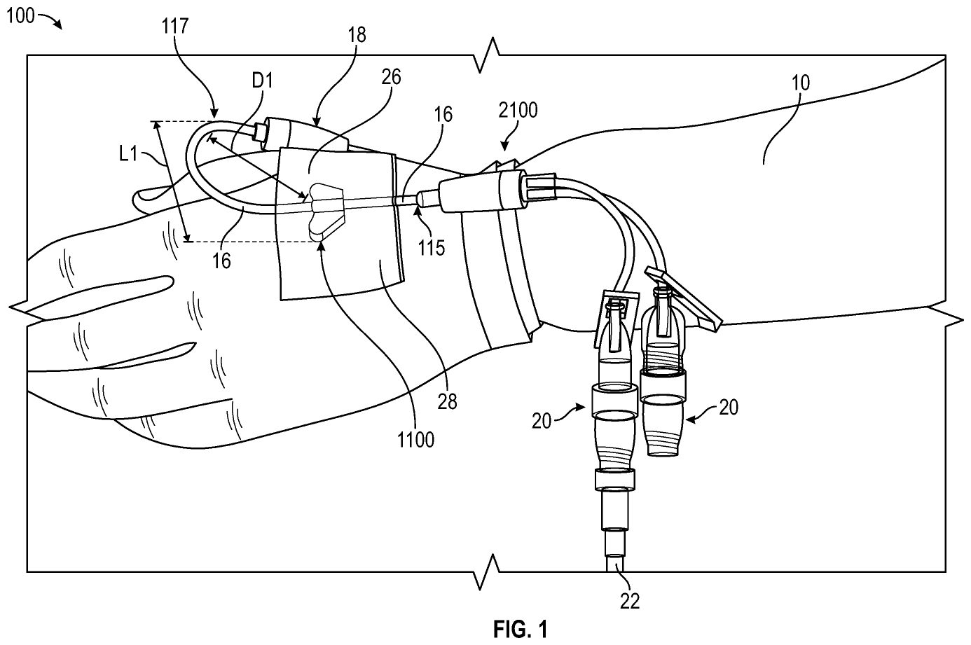

FIG. 1 illustrates a medical fluid extension set system 100 in accordance with aspects of the present disclosure. The medical fluid extension set system 100 includes tubing 16, a tubing anchor 1100, and a fluid coupling device 2100, where the tubing anchor 1100 is configured to resist unintended separation or dislodgment of the medical fluid extension set system 100 from the patient, and the fluid coupling device 2100 is configured to disconnect and obstruct the fluid pathway of the medical fluid extension set system.

The tubing 16 of the medical fluid extension set system has a first end 115 and a second end 117, and the tubing anchor 1100 is coupled to the tubing 16, between the first and second ends 115, 117 of the tubing.

The fluid coupling device 2100 is coupled to the first end 115 of the tubing 16 to form a fluid pathway between the medical fluid extension set system 100 and tubing 22 of an IV set. Although the fluid coupling device 2100 is illustrated as being coupled to tubing 22 of an IV set having first and second connectors 20, it should be understood that the present disclosure contemplates the fluid coupling device 2100 can be coupled to another fluid-transfer device or to tubing that extends from the fluid coupling device 2100 to another portion of the IV set, such as a medicament bag or IV pump.

The second end 117 of the tubing is illustrated as being coupled to a catheter 18 configured to form an intravenous fluid pathway with the patient. The second end 117 of the tubing can include a connector configured to removably couple with a catheter 18 or another device, and in some embodiments of the present disclosure, the second end 117 of the tubing is bonded or affixed to a catheter 18. Although the second end 117 of the tubing is illustrated as being coupled with a catheter 18, the present disclosure contemplates that the second end 117 of the tubing can be coupled with another fluid-transfer device or can extend directly into a patient, such as in the formation of an enteral feeding tube.

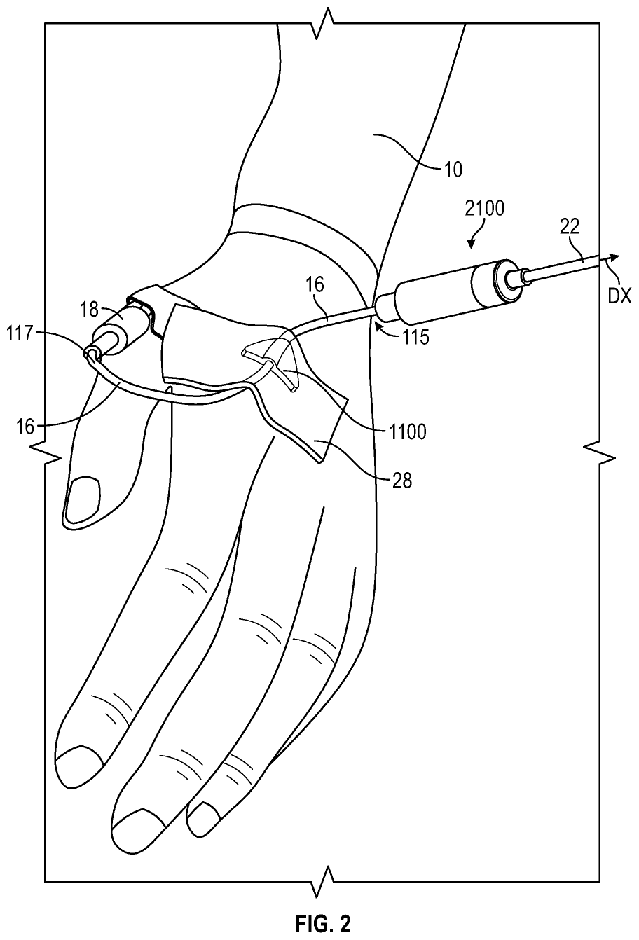

The tubing anchor 1100 is affixed to the patient with tape 28 that extends over a top surface of the tubing anchor 1100. Referring to FIG. 2, if the tubing 16 or another portion of the medical fluid extension set system, or a portion of the IV set, is pulled in a direction DX away from the tubing anchor or patient, engagement of the tape 28 against the tubing anchor 1100 and the patient 10 can resist the force and prevent unintended dislodgement or disconnection of the tubing anchor 1100 and/or and the catheter 18 from the patient.

The medical fluid extension set system 100 can be configured so that at least a portion of the tubing 16 forms a non-linear or curved shape to permit movement of the tubing anchor 1110 before movement of the second end 117 of the tubing. In some aspects of the present disclosure, the non-linear or curved shape portion of the tubing 16 can permit movement of the tubing anchor 1110 before movement of the second end 117 of the tubing when the medical fluid extension set system, or a portion of the IV set coupled thereto, is pulled in a direction DX away from the tubing anchor or the patient 10.

To form a non-linear or curved shape, the tubing anchor 1100 can be positioned a distance D1 from the second end 117 of the tubing, where the distance D1 is less than a length of the tubing extending between the tubing anchor 1100 and the second end 117 of the tubing. In some examples of the present disclosure, the distance D1 is less than the length of the tubing extending from the tubing anchor 1100 to the second end 117 of the tubing.

In some embodiments of the present disclosure, the medical fluid extension set system 100 is formed or manufactured with at least a portion of the tubing 16 having a non-linear or curved shape between the tubing anchor 1100 and the second end 117 of the tubing when the medical fluid extension set is unrestrained or unbiased. In some embodiments, the tubing 16, or a portion thereof, can include a bellows shape configured to permit elongation of the tubing 16, between the first and second ends 115, 117 of the tubing.

To resist or prevent separation of the medical fluid extension set system, or a portion thereof, from the patient, the medical fluid extension set system can include a fluid coupling device 2100 configured to disconnect and obstruct the fluid pathway of the medical fluid extension set system when the pulling or tension force exceeds a threshold. To disconnect and obstruct the fluid pathway of the medical fluid extension set system, the fluid coupling device 2100 includes a first valve assembly 2110 and a second valve assembly 2210 that are removably coupled together.

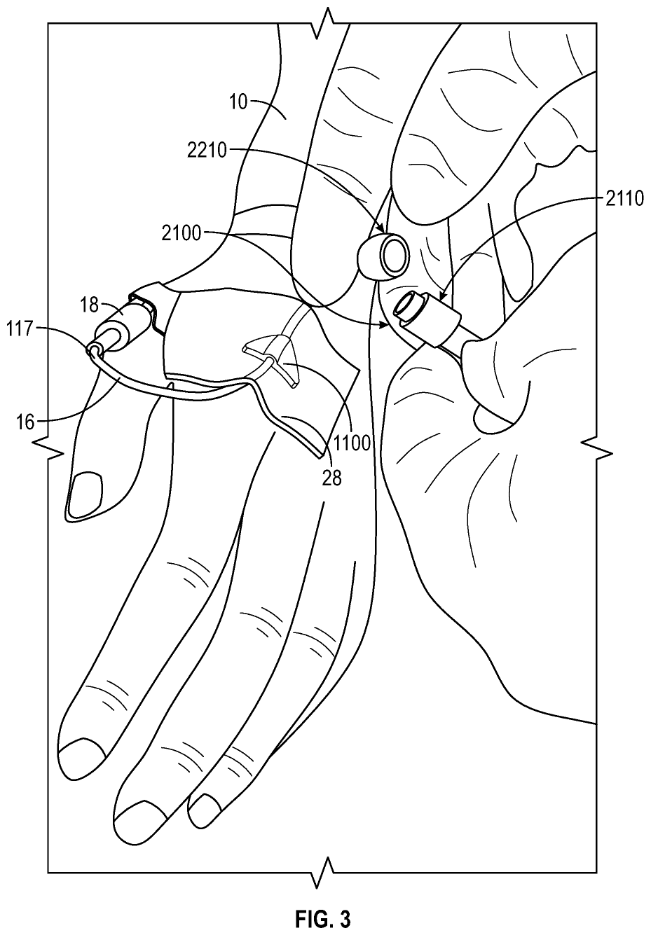

FIG. 3 illustrates the first and second valve assemblies 2110, 2210 in a disconnected configuration, such as after the tubing 22 of the IV set has been pulled in a direction away from the tubing anchor 1100 or patient 10 at a force that exceeded the disconnection threshold of the fluid coupling device 2100.

Upon disconnection or separation of the first valve assembly 2110 from the second valve assembly 2210, the fluid pathway of each of the first and second valve assemblies 2110, 2210 are obstructed. The obstructed fluid pathways prevents leakage of a fluid from the respective tubing 16, 22, coupled to the first and second valve assemblies 2110, 2210.

After disconnection or separation of the first valve assembly 2110 from the second valve assembly 2210, any of the first and second valve assemblies 2110, 2210 can be reconnected together to reestablish the fluid pathway with the medical fluid extension set system.

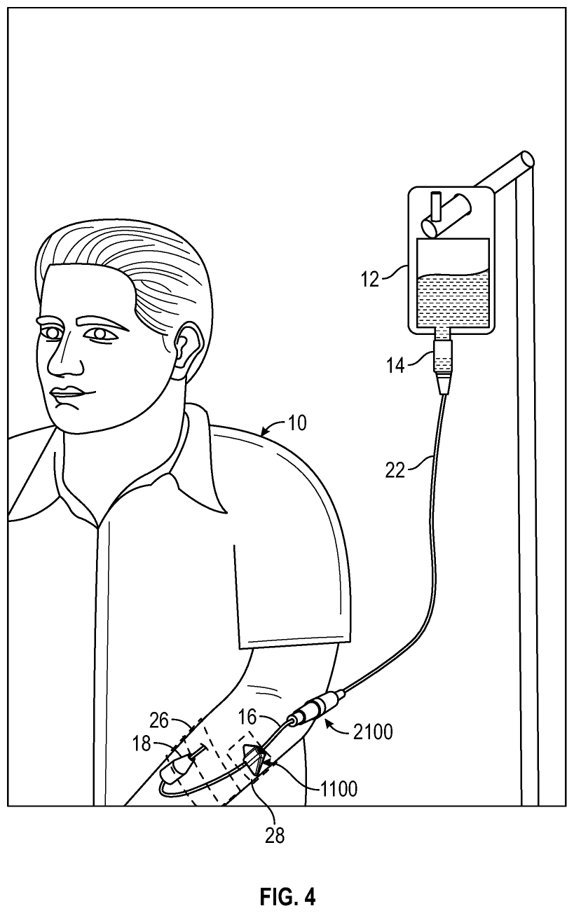

FIG. 4 illustrates the medical fluid extension set system 100 coupled to a patient and in use with an IV set. The IV set includes a medicament bag 12, a drip chamber 14, and tubing 22. The tubing 22 extends between the drip chamber 14 and the fluid coupling device 2100 of the medical fluid extension set system. It should be understood that the present disclosure contemplates that any of the first and second valve assemblies 2110, 2210 can be coupled to the tubing 22 of the IV set, and the other of the first and second valve assemblies 2110, 2210 can be coupled to the tubing 16 of the medical fluid extension set system.

To resist unintended dislodgement or disconnection of the tubing 16 or the catheter 18 from the patient, tape 26 is placed over the tubing 16 and the catheter 18, so that the tape engages the tubing 16, the catheter 18, and the patient. To increase the resistance to unintended dislodgement or disconnection of the tubing 16 or catheter 18, the tubing anchor 1100 is positioned along the tubing 16, and tape 28 is placed over the tubing anchor 1100, so that the tape engages the tubing anchor 1100 and the patient 10.

In use, if the tubing 16, 22, or another portion of the medical fluid extension set system 100 and IV set between the tubing anchor 1100 and the medicament bag 12, is pulled in a direction away from the tubing anchor, the tape 28, which is engaged against the tubing anchor 1100 and the patient 10, can resist the force and prevent unintended dislodgement or disconnection of tubing anchor 1100 and the catheter 18 from the patient.

In some embodiments of the present disclosure, tubing anchor 1100 can have tape and/or an adhesive along a surface configured to be engaged against a patient and configured to resist unintended separation of the tubing anchor 1100 from the anchor.

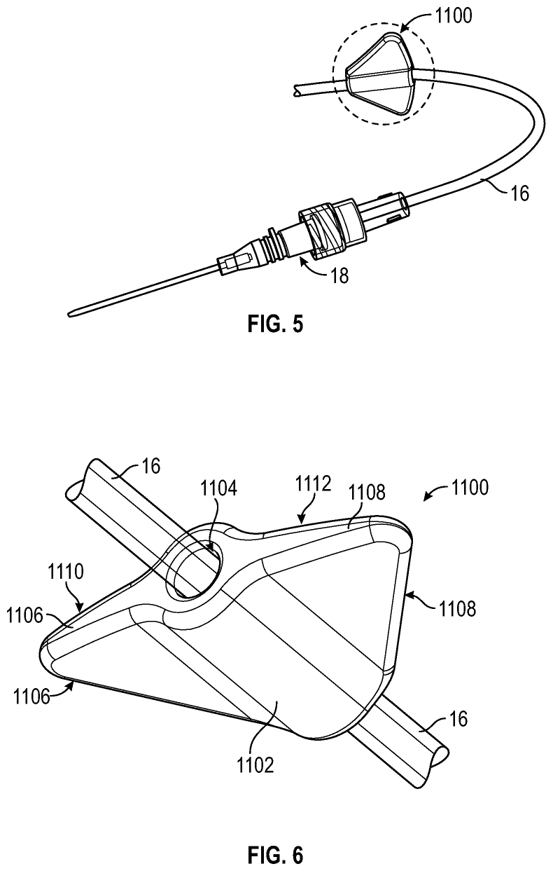

Referring now to FIG. 5, the medical fluid extension set system 100 is illustrated with a catheter 18 coupled to the second end 117 of the tubing, and without the fluid coupling device 2100 for clarity. A detailed view of the tubing anchor 1100 and a portion of the tubing 16 is illustrated in FIG. 6. The tubing anchor 1100 includes an anchor body with an inner surface forming a tubing passage and an outer surface forming a sidewall, where the sidewall extends in a direction away from the tubing passage. The outer surface of the anchor body can provide a larger surface area, relative to the surface area of the tubing along a coextensive length of the tubing anchor, for engagement by the tape. In some embodiments of the present disclosure, the tubing anchor 1100 can include first and second sidewalls that extend in a direction away from the tubing passage.

Although the tubing anchor 1100 is illustrated as being coupled along a fluid pathway of a medical fluid extension set, it should be understood that the tubing anchor 1100 can be connected to other fluid pathways, such as IV tubing, an IV set, or an enteral feeding tube. It should also be understood that the present disclosure contemplates embodiments in which a tubing anchor is positioned along another portion of a fluid pathway configured for coupling to a patient.

Various embodiments contemplated by the present disclosure can include, but are not limited to, embodiments in which a medical fluid extension set includes a fluid connector 20, a catheter 18, and a fluid coupling device 2100 coupled to the tubing 16. A tubing anchor 1100 can be coupled to the tubing 16 between the fluid connector 20 and the catheter 18. In some embodiments of the present disclosure, a tubing anchor 1100 can be coupled to the tubing 16 between the fluid coupling device 2100 and the catheter 18.

A tubing anchor 1100, in some embodiments of the present disclosure, is coupled to or integrated into a fluid coupling device 2100. The tubing anchor 1100 can be coupled to or integrated into a fluid coupling device 2100 such that the tubing anchor 1100 is movable, relative to the fluid coupling device 2100.

In some embodiments of the present disclosure, a medical fluid extension set can have a tubing anchor 1100 that is coupled to tubing 16, between a fluid connector 20 and a fluid coupling device 2100, and the tubing anchor 1100 is positioned proximal to the fluid connector 20 so that a distance between the tubing anchor 1100 and the fluid connector 20 is less than a distance between the tubing anchor 1100 and the fluid coupling device 2100. A medical fluid extension set can have a tubing anchor 1100 that is coupled to tubing 16, between a fluid connector 20 and a fluid coupling device 2100, and is positioned proximal to the fluid coupling device 2100 so that a distance between the tubing anchor 1100 and the fluid coupling device 2100 is less than a distance between the tubing anchor 1100 and the fluid connector 20.

In some embodiments of the present disclosure, a medical fluid extension set can have a tubing anchor 1100 that is coupled to tubing 16, between a fluid coupling device 2100 and a fluid connector 20 (e.g., upstream of the fluid coupling device 2100). A medical fluid extension set can have a tubing anchor 1100 that is coupled to a patient 10 such that the tubing 16 is between the patient's body or skin and the tubing anchor 100. A medical fluid extension set can have a first tubing anchor 1100 that is coupled to a first length of the tubing 16, where the first length the tubing 16 is coupled to a first valve assembly 2110 of the fluid coupling device, and a second tubing anchor 1100 that is coupled to a second length the tubing 16, where the second length the tubing 16 is coupled to a second valve assembly 2210 of the fluid coupling device, such that the fluid coupling device 2100 is positioned between the first and second anchors 1100.

An embodiment of the tubing anchor 1100 is illustrated in FIG. 6. The tubing anchor 1100 includes an anchor body 1102 with an inner surface that forms a tubing passage 1104 extending through the anchor body 1102. The tubing passage 1104 can have a cross-sectional diameter that is approximately equal to or greater than a diameter defined by the outer surface of the tubing 16.

The anchor body 1102 also includes a first sidewall 1106 and a second sidewall 1108, where each of the first and second sidewalls 1106, 1108 extend in a direction away from the tubing passage 1104. In some embodiments of the present disclosure, the first and second sidewalls 1106, 1108 are shaped to form first and second wings 110, 1112, respectively.

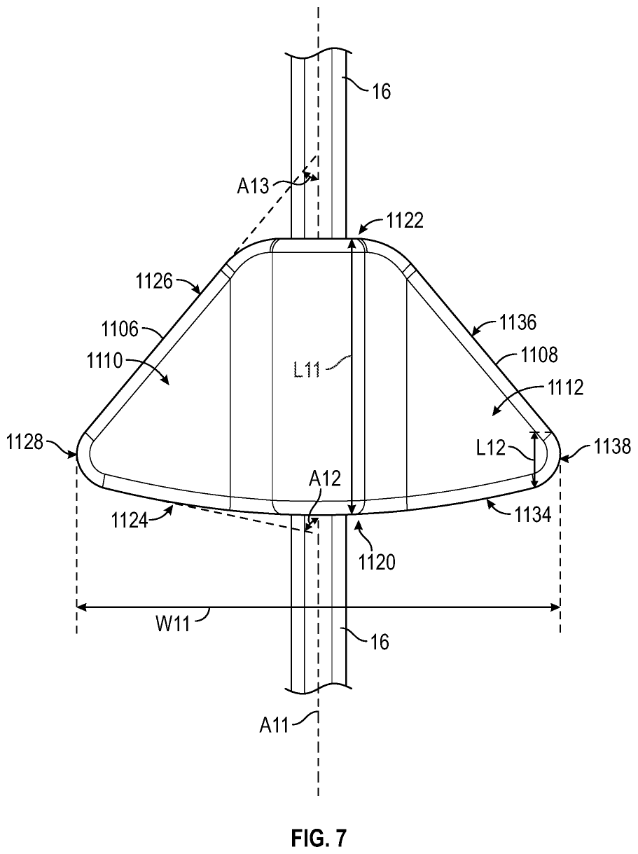

Referring to the top elevation view of the tubing anchor 1100 in FIG. 7, the tubing passage 1104 extends through a first end 1120 and a second end 1122 of the anchor body and defines a longitudinal axis A11 through the anchor body. The first and second sidewalls 1106, 1108 extend in a direction away from the longitudinal axis A11. The first sidewall 1106 extends in a first direction away from the longitudinal axis A11, and the second sidewall 1108 extends in a second direction away from the longitudinal axis A11, where the second direction is different than the first direction.

The first sidewall 1106 also extends between the first end 1120 and the second end 1122 of the anchor body, and the second sidewall 1108 extends between the first end 1120 and the second 1122 of the anchor body. The first sidewall 1106 includes a front portion 1124 and a rear portion 1126, where the front portion 1124 extends away from the first end 1120, and the rear portion 1126 extends away from the second end 1122. The front and rear portions 1124, 1126 intersect at an apex 1128 of the first sidewall.

The second sidewall 1108 includes a front portion 1134 and a rear portion 1136, where the front portion 1134 extends away from the first end 1120, and the rear portion 1136 extends away from the second end 1122. The front and rear portions 1134, 1136 intersect at an apex 1138 of the second sidewall.

The front portions 1124, 1134 and the rear portions 1126, 1136 of the first and second sidewalls 1106, 1108, respectively, can extend toward each other to form a wing shape that tapers away from the longitudinal axis A11. The wings 1110, 1112 can be configured with a proximal portion adjacent to the longitudinal axis A11 and an apex that is distal to the longitudinal axis A11. In embodiments in which the wing shape that tapers away from the longitudinal axis A11, the anchor body can have a first length L12, adjacent the tubing passage, that decreases in a direction away from the longitudinal axis A11 toward a second length L12.

In some aspects of the present disclosure, the front portions 1124, 1134 of the first and second sidewalls 1106, 1108, respectively, extends away from the longitudinal axis A11 at a first angle A12 therebetween, and the rear portions 1126, 1136 of the first and second sidewalls 1106, 1108, respectively, extends away from the longitudinal axis A11 at a second angle A13 therebetween. The first and second angles A12, A13 can be selected such that the front and rear portions of the sidewalls 1106, 1108 intersect. In some embodiments, the first angle A12 is greater than the second angle A13.

Although the tubing anchor 1100 is illustrated having an anchor body 1102 with first and second sidewalls 1106, 1108 forming first and second wings, it should be understood that in some embodiments of the present disclosure an anchor body 1102 having one sidewall or more than two sidewalls is contemplated. In some embodiments of the present disclosure, it is also contemplated that the wings can be configured to have a length that increases in a direction away from the longitudinal axis A11.

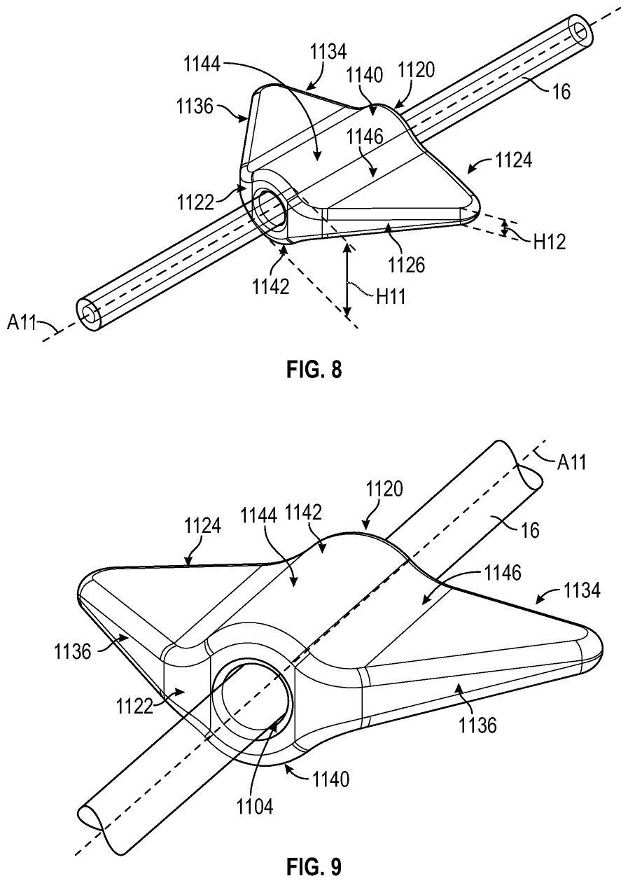

The anchor body 1102 further defines a top end 1140 and a bottom end 1142 of the tubing anchor, as illustrated in FIGS. 8 and 9. The top end 1140, illustrated in FIG. 8, can include at least a portion of a top surface 1141 of the tubing anchor, and the bottom end 1142, illustrated in FIG. 9, can include at least a portion a bottom surface 1143 of the tubing anchor.

Any of the top and bottom surfaces of the tubing anchor 1100 can form any of a convex and/or concave shape. The top surface can form a convex shape 1144 that extends along the tubing passage, between the first and second ends 1120, 1122 of the anchor body. A portion of the top surface can also form a concave shape 1146 that is adjacent to the tubing passage. The convex shape 1144 can extend between the first and second ends 1120, 1122 of the anchor body, and be positioned between portions of the outer surface that have a concave shape 1146.

The bottom surface can form a profile shape with convex and concave shapes that mirror the top surface. In some embodiments of the present disclosure, the top and bottom surfaces can have the same or different profile shapes. For example, the top surface can include a convex shape and the bottom surface can form a concave shape, where the convex shape of the top surface can provide for an anchor body with a low profile to avoid being bumped or pulled unintentionally while providing a large surface area relative to the height of the anchor body. The concave shape of the bottom surface that corresponds with a convex shaped surface defined by a patient's body thereby increasing patient comfort when the anchor body is engaged against the patient.

The top and bottom ends 1140, 1142 of the anchor body 1102 define a height H11 of the anchor body therebetween, and the apex 1128, 1138 of the first and second sidewalls define a width W11 of the anchor body therebetween. The width W11 can be greater than the height H11 of the anchor body, where the width W11 and length L12 can be configured to provide a larger surface are for engaging the tubing anchor against a length of tape and against the patient.

In some embodiments, the height of the anchor body decreases in a direction away from the longitudinal axis A11. For example, a concave portion of the outer surface of the anchor body can form a portion the height H11 that tapers away from the longitudinal axis A11. In some embodiments, the width of the anchor body decreases between the first and second ends 1120, 1122. For example, the width of the anchor body can decrease in a direction from the first end 1120 toward the second end 1122, thereby providing a wider cross-sectional profile of the first end 1120, relative to the second end 1122. The wider cross-sectional profile of the first end 1120 can provide resistance to unintentional separation of the tubing from the patient when the tubing anchor 1100 is pulled in a direction from the second end 1122 toward the first end 1120.

The anchor body 1102, or portion thereof, can be formed from a soft plastic, such as a thermoplastic elastomer (TPE). In some embodiments, a portion of the anchor body 1102 can be more flexible, relative to another portion thereof. For example, a portion of the anchor body 1102 proximal to an apex of a sidewall can have a height H12 that is less than the H11, such that the portion of the anchor body 1102 proximal to an apex of a sidewall is more flexible relative to the portion of the anchor body 1102 having the height H11. The flexible portion of the anchor body 1102 can be configured to be biased or flexed relative to the skin of a patient to more closely correspond to the patient's body.

The tubing anchor 1100 can be coupled to a length of tubing by inserting the tubing through the tubing passage 1104. The tubing anchor 1100 can then be moved or slid along the tubing 16 to a desired position. The tubing anchor 1100 can then be affixed to the patient by taping the tubing anchor 1100 to the patient or by exposing an adhesive on an outer surface of the tubing anchor. The adhesive can extend along a bottom surface of the tubing anchor 1100 that is configured to engage against the patient.

The medical fluid extension set system 100 can configured so that longitudinal movement of the tubing anchor 1100 along the tubing 16, between the first and second ends 115, 117 of the tubing is resisted. To resist movement of the tubing anchor 1100 along the tubing 16, the tubing anchor 1100 can be affixed to the tubing 16 by adhering the tubing anchor 1100 and tubing 16 together, such as by an adhesive or weld between an outer surface of the tubing 16 and the inner surface that forms a tubing passage 1104. In some embodiments of the present disclosure, movement of the tubing anchor 1100 along the tubing 16 can be resisted by an interference fit between the tubing anchor 1100 and the tubing 16. To form the interference fit, the tubing passage 1104 can have a cross-sectional diameter that is approximately equal to or less than a diameter defined by the outer surface of the tubing 16.

The tubing anchor 1100 can be coupled with the tubing 16 so that any of the first end 1120 or the second end 1122 of the anchor body is oriented toward the fluid coupling device 2100. In some embodiments of the present disclosure, the second end 1122 of the tubing anchor is oriented toward the first end 115 of the tubing having the fluid coupling device. In some aspects of the present disclosure, the second end 1122 of the tubing anchor provides a greater surface area, relative to the first end 1120 of the tubing anchor, to resist a pulling force in the tubing 16 in a direction away from the tubing anchor 1100.

When another portion of the tubing spaced apart from the tubing anchor 1100, or another medical device coupled to the first end 115 of the tubing, is pulled, engagement of the tape or adhesive against the tubing anchor 1100 can resist separation of the tubing from the patient. In some instances, the tubing anchor 1100 can permit another portion of the medical fluid extension set system, such as the fluid coupling device 2100 to deflect or move relative to the tubing anchor 1100.

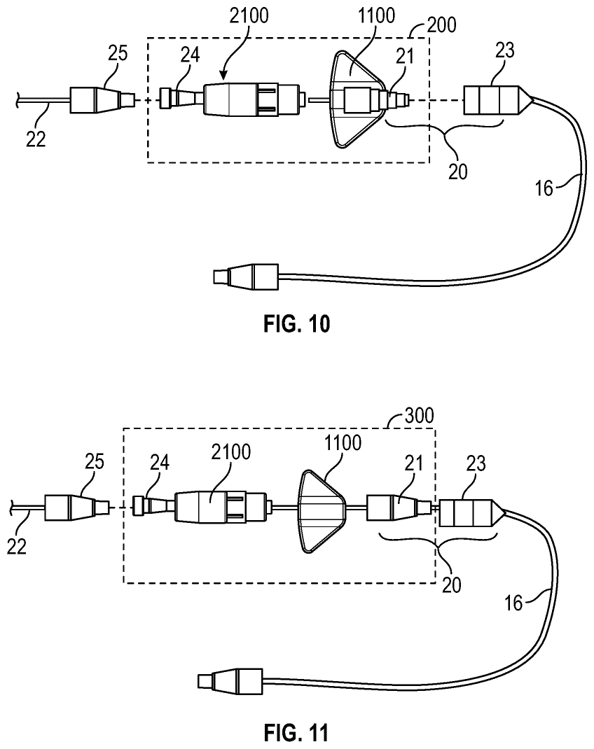

FIGS. 10 through 17 illustrate additional embodiments of a medical fluid extension set system having at least some features described in the present disclosure with reference to the medical fluid extension set system 100. Features of the embodiments disclosed or with reference to FIGS. 10 through 17, and which are consistent features described in other portions of the present disclosure, include like reference numbers for the sake of clarity and brevity.

The additional embodiments include, but are not limited to, medical fluid extension set systems where a tubing anchor is attached to tubing at a location that is upstream from a fluid connector 20. The fluid connector 20 can be configured as a connector having first and second portions that are separable, and where the first and second portions can form any of a needle-free connector and/or a luer connection therebetween. The first portion of the fluid connector can be formed as a male luer connector, and the second portion of the fluid connector can be formed as a needle-free connector, where the first and second portions can be removably coupled together. In some embodiments of the present disclosure, any of the first or second portions of the fluid connector can be formed as a female luer connector. In some instances of the present disclosure, a medical fluid extension set systems can include more than one fluid connector 20.

FIGS. 10 and 11 illustrate embodiments of a medical fluid extension set system 200, 300 that include a tubing anchor 1100 and a fluid coupling device 2100, where the tubing anchor 1100 is fluidly coupled to tubing at a location upstream from a portion of a fluid connector 20.

To fluidly couple the medical fluid extension set systems 200, 300 upstream from a portion of a fluid connector, the medical fluid extension set systems 200, 300 are configured to with a fluid coupling device 2100 and a first portion 21 of a fluid connector that are fluidly coupled together.

The first portion 21 of the fluid connector is configured to be fluidly coupled to a second portion 23 of the fluid connector, such that the tubing anchor 1100 is upstream from the second portion 23 of the fluid connector. For example, when the medical fluid extension set system 200, 300 is coupled between an IV set and a catheter, the tubing anchor 1100 is upstream from the second portion 23 of the fluid connector and the catheter is downstream of the second portion 23 of the fluid connector.

Referring to FIG. 10, the medical fluid extension set system 200 is configured with the tubing anchor 1100 coupled to or integrated into the first portion 21 of a fluid connector. In FIG. 11, the medical fluid extension set system 300 is illustrated with the tubing anchor 1100 spaced apart from the first portion 21 of a fluid connector.

The medical fluid extension set systems 200, 300 can reduce or prevent the fluid connector, or a portion of the fluid connector 21, 23 from causing a pressure point against the patient's body. The medical fluid extension set system 200, having an integrated tubing anchor 1100 and first portion 21 of a fluid connector, can provide a reduced length for the medical fluid extension set system 200 relative to a medical fluid extension set system having a tubing anchor 1100 that is spaced apart from the first portion 21 of a fluid connector.

In some embodiments of the present disclosure, the fluid coupling device 2100 can be coupled to tubing 22 of an IV set, and the second portion 23 of the fluid connector can be coupled to a catheter so that the medical fluid extension set systems 200, 300 is fluidly coupled between the IV set and the catheter.

To fluidly couple the medical fluid extension set systems 200, 300 between an IV set and a catheter, the medical fluid extension set systems 200, 300 includes a second portion 24 of a fluid connector upstream from the fluid coupling device 2100. The second portion 24 of the fluid connector can be coupled to a first portion 25 of a fluid connector of the IV set. As such, the medical fluid extension set system 200, 300 can be separated or disconnected from between the first portion 25 of a fluid connector of the IV set and the second portion 23 of the fluid connector. In some aspects of the present disclosure, when the medical fluid extension set system 200, 300 is separated or disconnected from between the IV set and the second portion 23 of the fluid connector, the first portion 25 of a fluid connector of the IV set can be directly coupled to the second portion 23 of the fluid connector.

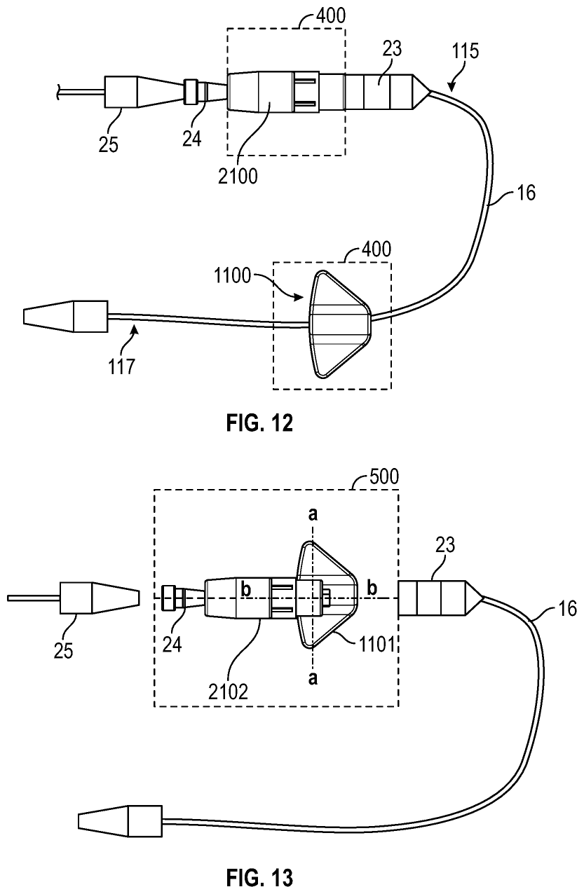

In some embodiments of the present disclosure, the tubing anchor 1100 and the fluid coupling device 2100 can be separately couplable to tubing 16 between tubing 22 of an IV set and a catheter. An example of a medical fluid extension set system 400 with a fluid coupling device 2100 and a tubing anchor 1100 fluidly coupled together by tubing 16 is illustrated in FIG. 12.

A first end 115 of the tubing is coupled to the second portion 23 of a fluid connector, and the second portion 23 of a fluid connector is configured to fluidly couple with the fluid coupling device 2100. The second end 117 of the tubing is configured to fluidly couple with a catheter or another portion of a fluid pathway. The tubing anchor 1100 is coupled to the tubing, between the first and second ends 115, 117 of the tubing.

The configuration of the medical fluid extension set system 400 provides for the fluid coupling device 2100 to be positioned upstream from the second portion 23 of the fluid connector, and the tubing anchor 1100 and the catheter to be downstream from the second portion 23 of the fluid connector. The features of the medical fluid extension set system 400 also provide for a reduced length of the system 400 relative to a medical fluid extension set system having a tubing anchor 1100 that is positioned between the fluid coupling device 2100 and the second portion 23 of a fluid connector.

In some aspects of the present disclosure, the medical fluid extension set system 400 can be coupled to an existing IV set by disconnecting upstream and downstream portions of a fluid connector, e.g., disconnecting a second portion 24 of a fluid connector from a second portion 23 of another fluid connector. The fluid coupling device 2100 can be inserted and coupled between the upstream and downstream portions of the fluid connector. The tubing anchor 1100 can be coupled to a length of the tubing that is downstream from the second portion 23 of a fluid connector, e.g., between the second portion 23 of a fluid connector and the catheter.

In any embodiment of the present disclosure, a tubing anchor can be coupled to or integrated with a fluid coupling device, such that the tubing anchor and fluid coupling device are movable, relative to each other. FIG. 13 illustrates an example of an embodiment of a medical fluid extension set system 500 having a tubing anchor 1101 coupled to or integrated with a fluid coupling device 2102.

The tubing anchor 1101 and the fluid coupling device 2102 are coupled together so that any of the tubing anchor 1101 and the fluid coupling device 2102 are movable in one or more direction, relative to each other. The movement between the tubing anchor 1101 and the fluid coupling device 2102 can be a rotational movement in at least one direction or along at least one plane.

To permit movement between the tubing anchor 1101 and the fluid coupling device 2102, the medical fluid extension set system 500 can include a structure forming a joint or hinge between the tubing anchor 1101 and the fluid coupling device 2102. In some embodiments of the present disclosure, any of the tubing anchor 1101 and the fluid coupling device 2102 can include a slot, channel, or groove configured to receive a portion of a ridge or post of the other of the tubing anchor 1101 and the fluid coupling device 2102, such that the tubing anchor 1101 and the fluid coupling device 2102 can pivot, rotate, or slide, relative each other. In any embodiment of the present disclosure, one or more structure joining or coupling the tubing anchor 1101 and the fluid coupling device 2102 together can form pivot coupling between the tubing anchor 1101 and the fluid coupling device 2102.

The movement between the tubing anchor 1101 and the fluid coupling device 2102 can be relative to an axis a-a extending through the fluid coupling device 2102 and the tubing anchor 1101. The fluid coupling device 2102 can pivot around the tubing anchor 1101 by rotating the fluid coupling device 2102 around the axis a-a. In some embodiments, a direction of movement between the tubing anchor 1101 and the fluid coupling device 2102 can be relative to a longitudinal axis b-b extending through opposing ends of the fluid coupling device 2102, where longitudinal axis b-b is transverse, relative to the longitudinal axis a-a.

When the medical fluid extension set system 500 is coupled to a patient with the tubing anchor 1101 engaged against or affixed to the patient, pivoting the fluid coupling device 2102 around the axis a-a results in the fluid coupling device 2102 rotating around the axis a-a such that the fluid coupling device 2102 moves away from or toward the patient. In embodiments in which a pivot coupling is configured for movement of the fluid coupling device 2102 around the longitudinal axis b-b, the fluid coupling device 2102 can be rotated or twist around the longitudinal axis b-b.

The movement between the tubing anchor 1101 and the fluid coupling device 2102 can resist bending loads from being transmitted to the anchor, which could otherwise act to detach the anchor from of the patient. For example, if tubing 22 of an IV set coupled to the fluid coupling device 2102, or the fluid coupling device 2102 itself, is pulled in a direction away from the patient, the fluid coupling device 2102 can pivot or rotate around the axis a-a and in a direction away from the patient in the manner of a lever rotating relative to a fulcrum. In another example, the fluid coupling device 2102 can rotate around the longitudinal axis b-b, which can resist transmitting a torsional stress to the tubing anchor 1101, such as when tubing 22 of an IV set coupled to the fluid coupling device 2102, or the fluid coupling device 2102 itself, is twisted.

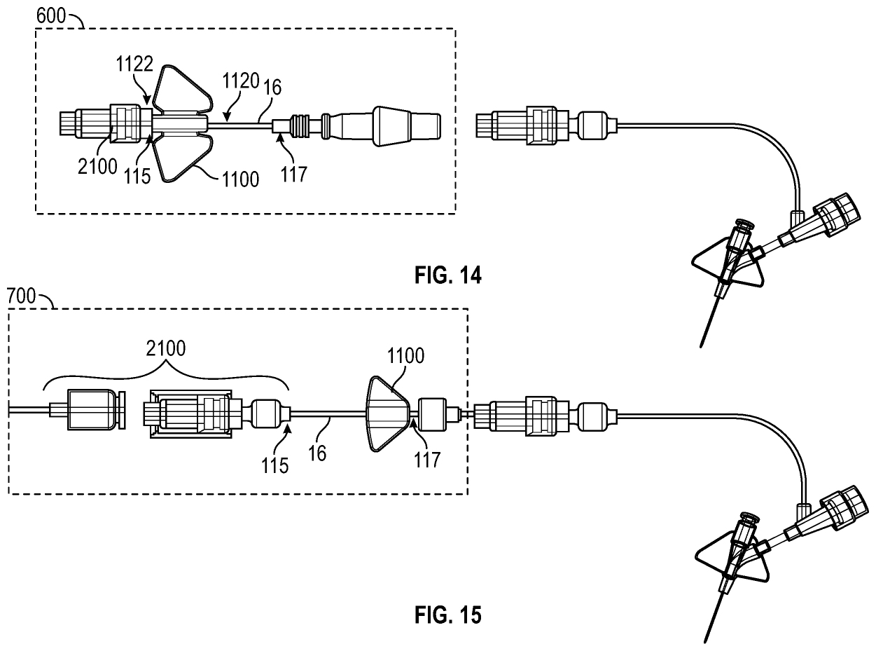

Referring to FIGS. 14 and 15, embodiments of medical fluid extension set systems 600, 700 are illustrated having a fluid coupling device 2100 fluidly coupled to a length the tubing 16, and a tubing anchor 1100 coupled to the tubing 16.

FIG. 14 illustrates an embodiment of the medical fluid extension set system 600 in which the tubing anchor 1100 and the fluid coupling device 2100 are positioned at a distal end of the medical fluid extension set system 600. To position the tubing anchor 1100 and the fluid coupling device 2100 at the distal end of the medical fluid extension set system, a first end 115 of the tubing is fluidly coupled to the fluid coupling device 2100, and the tubing anchor 1100 is coupled to a portion of the tubing 16 that is adjacent to and extends from the first end 115 of the tubing.

In some embodiments of the present disclosure, the first end 115 the tubing is coupled to any of a first end 1120 or a second end 1122 of the tubing anchor 1100, and the other of the first end 1120 or the second end 1122 of the tubing anchor 1100 is coupled to the fluid coupling device 2100. When the tubing is coupled to the tubing anchor 1100, the tubing passage 1104 of the tubing anchor can form a portion of a fluid pathway therethrough.

FIG. 15 illustrates an embodiment of the medical fluid extension set system 700 in which the tubing anchor 1100 and the fluid coupling device 2100 are spaced apart along tubing 16, such that the tubing anchor 1100 is positioned at a distal end, and the fluid coupling device 2100 is positioned at a proximal end of the medical fluid extension set system 700. To position the tubing anchor 1100 and the fluid coupling device 2100 at proximal and distal ends of the medical fluid extension set system, respectively, a first end 115 of the tubing is fluidly coupled to the fluid coupling device 2100, and the tubing anchor 1100 is coupled to a portion of the tubing 16 that is adjacent to and extends from the second end 117 the tubing.

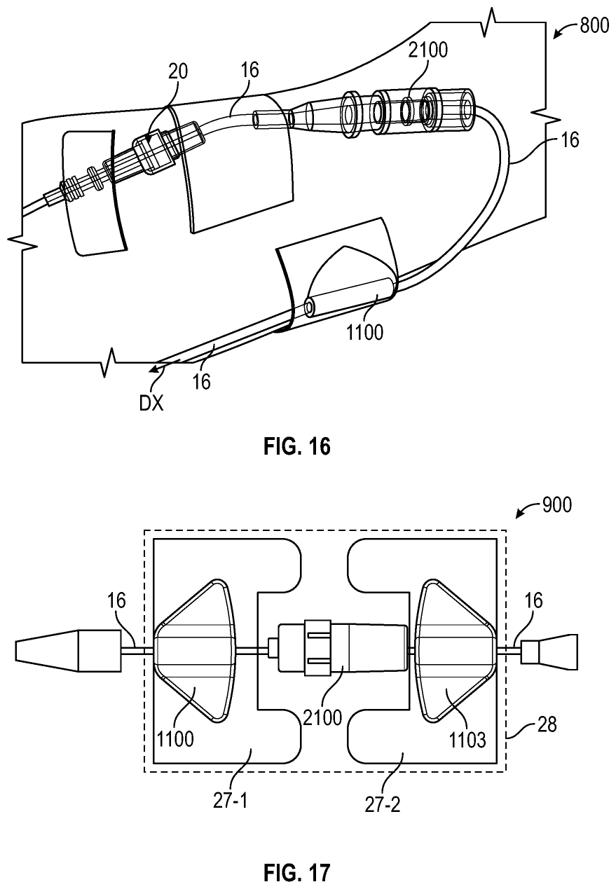

FIG. 16 illustrates an embodiment of the medical fluid extension set system 800 in which a tubing anchor 1100 is positioned upstream from a fluid coupling device 2100 such that the fluid coupling device 2100 is positioned between a catheter and the tubing anchor 1100.

The medical fluid extension set system 800 is configured so that the tubing anchor 1100 can resist separation of the medical fluid extension set system 800 from the patient when the tubing 16, or another portion of the medical fluid extension set system 800 or a portion of the IV set, is pulled in a direction DX away from the tubing anchor or patient. However, if the tubing anchor becomes separated from the patient due to a force caused by the pulling, the fluid coupling device 2100 is configured to disconnect the fluid pathway. Thus, in some embodiments of the present disclosure, the disconnection force of the fluid coupling device 2100 is less than the securement force of the tubing anchor 1100 to the skin of the patient. In some aspects of the present disclosure, the disconnection force of the fluid coupling device 2100 is less than the securement for of the catheter to the patient. In some embodiments, the disconnection force of the fluid coupling device 2100 is approximately 1 to 2 lbs.

Features of the medical fluid extension set system 800 can reduce the occurrence of disconnection events for forces that are less than the tubing anchor 1100 securement strength, which could ensure that the fluid coupling device 2100 only separates after the first securement, e.g., the tubing anchor 1100, becomes separated from the patient.

To provide controlled support of the fluid coupling device 2100, or a portion thereof, after the tubing anchor 1100 becomes separated from the patient, a medical fluid extension set system 900 can be configured with a fluid coupling device 2100 positioned between a first tubing anchor 1100 and a second tubing anchor 1103, as illustrated in FIG. 17.

The medical fluid extension set system 900 can include a securement adhesive configured to affix the first and second tubing anchors 1100 and 1103 to the patient. The securement adhesive can be shaped with a cutout or having a shape forming a window so that the securement adhesive does not extend between the patient and the fluid coupling device 2100. In some aspects of the present disclosure, a first securement adhesive 27-1 and a second securement adhesive 27-2 are provided for affixing the first tubing anchor 1100 and the second tubing anchor 1103 to the patient, respectively.

To maintain the position of the first and second securement adhesives relative to each other, the medical fluid extension set system 900 can include a film overlay 28 configured to extend over at least the first and second tubing anchors 1100 and 1103.

The medical fluid extension set system 900 is configured so that the second tubing anchor 1103 can separate from the patient before the first tubing anchor 1100. To permit the second tubing anchor 1103 to separate from the patient before the first tubing anchor 1100, the second tubing anchor 1103 can be smaller than the first tubing anchor 1100. Upon separation of the second tubing anchor 1103, first and second portions of the fluid coupling device 2100 can separate to disconnect the fluid pathway between the first and second tubing anchors 1100, 1103. After the fluid coupling device 2100 becomes separated, the first securement adhesive 27-1 can retain the first tubing anchor 1100 coupled to the patient.

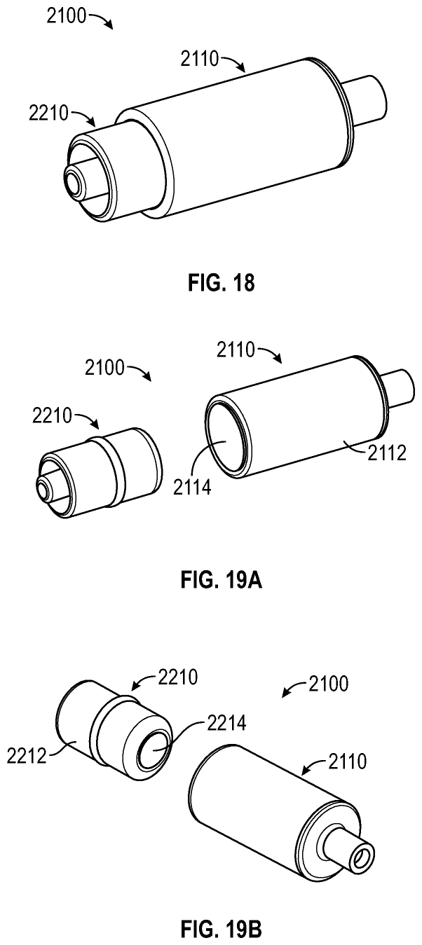

To permit disconnection of the fluid pathway after separation of the second tubing anchor 1103 and before separation of the first tubing anchor 1100, the disconnection force of the fluid coupling device 2100 is less than the securement force of the tubing anchor 1100 to the skin of the patient. In some embodiments, the disconnection force of the fluid coupling device 2100 is approximately 1 to 2 lbs. Referring now to the FIG. 18, the fluid coupling device 2100 is illustrated in accordance with aspects of the present disclosure. The fluid coupling device 2100 includes a first valve assembly 2110 and a second valve assembly 2210. The first and second valve assemblies 2110, 2210 can be coupled together by inserting a portion of the second valve assembly 2210 into the first valve assembly 2110. When the first valve assembly 2110 and the second valve assembly 2210 are coupled together, a fluid pathway is formed through the fluid coupling device 2100.

If any of the first valve assembly 2110 and the second valve assembly 2210 are pulled away from each other, such as when a pulling or tension force on the fluid coupling device 2100 exceeds a threshold, the first valve assembly 2110 and the second valve assembly 2210 can separate from each other. When the first and second valve assemblies 2110, 2210, separate from each other, as illustrated in FIGS. 19A and 19B, the respective first and second valve assemblies 2110, 2210 can resist a fluid flow therethrough. In some embodiments of the present disclosure, each of the first and second valve assemblies 2110, 2210 can resist fluid flow by obstructing a fluid passage through their respective assemblies.

The first valve assembly 2110 includes a housing 2112 with an inner cavity and a compressible valve 2114 positioned in the cavity. When the first valve assembly 2110 is not coupled with the second valve assembly 2210 or other mating connector, the compressible valve 2114 is in a first position, wherein a fluid passage through the first valve assembly 2110 is obstructed by the compressible valve 2114 to resist fluid flow through the fluid passage of the first valve assembly 2110.

When the first valve assembly 2110 is coupled with the second valve assembly 2210, the compressible valve 2114 is moved to a second position, wherein the fluid passage through the first valve assembly 2110 is unobstructed to reduce the resistance to a fluid flow through the fluid passage of the first valve assembly 2110.

The second valve assembly 2210 includes a body 2212 with an inner bore and a valve plug 2214 positioned in the bore 2220. When the first valve assembly 2110 is not coupled with the second valve assembly 2210, a fluid passage through the second valve assembly 2210 is obstructed by the valve plug 2214.

When the first and second valve assemblies 2110, 2210 are coupled together, a portion of the first valve assembly 2110 moves or biases the valve plug 2214 to fluidly couple the fluid passage through the first valve assembly 2110 with the fluid passage through the second valve assembly 2210.

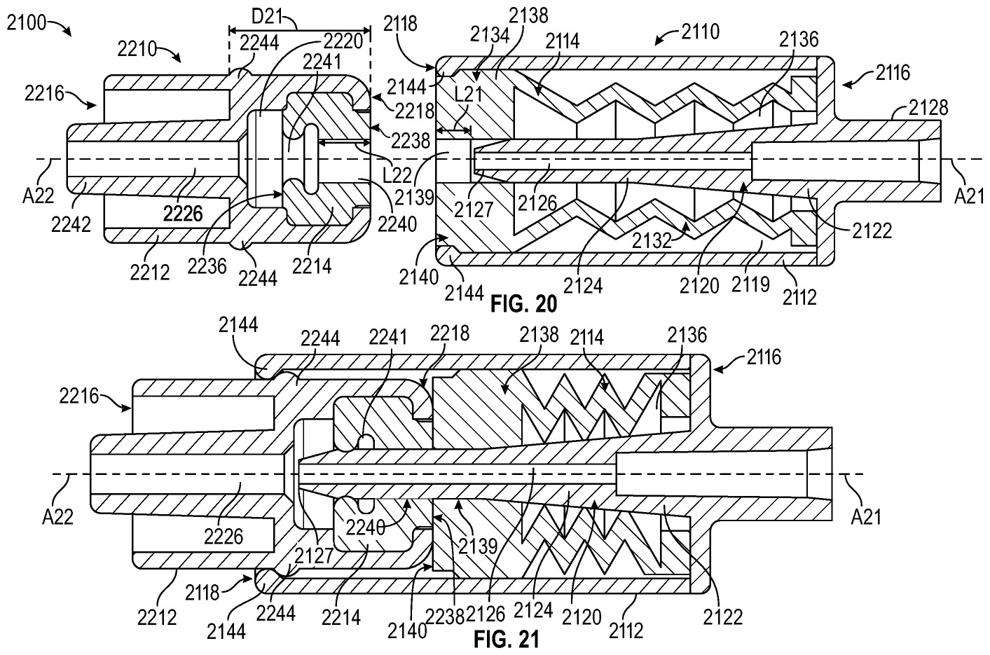

A cross-sectional view of the fluid coupling device 2100 is illustrated in FIGS. 20 and 21. The first valve assembly 2110 includes a housing 2112 having a first end 2116 and a second end 2118. An inner surface of the housing 2112 form a cavity 2119 that at extends through the second end 2118 toward the first end 2116 of the housing.

The first valve assembly 2110 also includes a post 2120 that extends within the cavity and forms at least a portion of a fluid passage through the first valve assembly 2110. The post includes a proximal end portion 2122 and a distal end portion 2242124, where the distal end portion 2242124 of the post extends within the cavity in a direction from the first end 2116 of the housing toward the second end 2118 of the housing. In some embodiments of the present disclosure, the proximal end portion 2122 of the post is coupled with the first end 2116 of the housing such that the fluid passage 2126 of the first valve assembly 2110 extends through the first end 2116 of the housing and the post 2120.

The post 2120 includes an inner surface forming a fluid passage 2126 that extends through the proximal and distal end portions 2122, 2242124 of the post. At the distal end portion 2242124 of the post, the fluid passage 2126 extends through an opening at a distal end 2127 of the post. In some embodiments of the present disclosure, the opening for the fluid passage is positioned at a location that is spaced apart from the distal end 2127 of the post.

In some embodiments of the present disclosure, the post 2120 can be configured as a needle that extends within the cavity and forms at least a portion of a fluid passage through the first valve assembly 2110. The post 2120 can be formed unitarily with the housing 2112 or another portion of the first valve assembly 2110, or the post 2120 can be coupled with the housing 2112 or another portion of the first valve assembly 2110. In some embodiments, the post 2120 is formed from a material that includes any of a polymer and/or a metal.

In some aspects of the present disclosure, the housing 2112 defines a boss 2128 that extends from the first end 2116 of the housing in a direction away from the second end 2118 of the housing. The boss 2128 can include a portion of the fluid passage 2126 of the first valve assembly 2110. A cross-sectional width of the fluid passage 2126 that extends through the boss 2128 can be configured as a bond pocket such that and end of a tubing can be positioned or inserted into the fluid passage 2126. In some embodiments of the present disclosure, the cross-sectional width of the fluid passage 2126 that extends through the boss 2128 is approximately equal to or greater than a cross-sectional width of the tubing. In some embodiments, tubing can be coupled to the first valve assembly 2110 using any of an interference fit and/or creating a bond between the first valve assembly 2110 and the tubing.

The compressible valve 2114 is positioned in the cavity and configured to resist fluid flow through the fluid passage 2126 when the compressible valve 2114 is in a first position. A fluid flow through the fluid passage 2126 is resisted by a portion of the compressible valve 2114 positioned between the distal end 2127 of the post and the second end 2118 of the housing when the compressible valve 2114 is in the first position.

The compressible valve 2114 includes a proximal end portion 2132 and a distal end portion 2134. The proximal end portion 2132 comprises a resilient member having an inner surface forming a recess 2136. The distal end portion 2134 comprises a head 2138 defining a distal end 2140 of the compressible valve 2114. The head 2138 includes a slit 2139 that extends through the head 2138 to the recess 2136.

Although the portion of the compressible valve 2114 that forms the resilient member is illustrated as a tube shaped structure having an accordion shaped wall when viewed in cross-section, the resilient member can be formed as any structure that can bias or direct the head 2138 toward the second end 2118 of the housing. In some embodiments of the present disclosure, the resilient member can be formed by a spring or arms that are positioned between the head 2138 and any of the first or second ends 2116, 2118 of the housing.

When the compressible valve 2114 is in the first position, the distal end 2140 of the compressible valve 2114 is aligned with the second end 2118 of the housing. In some embodiments of the present disclosure, a common plane intersects the distal end 2140 of the compressible valve 2114 and the second end 2118 of the housing when the compressible valve 2114 is in the first position.

To resist fluid flow through the fluid passage 2126, the first valve assembly 2110 is also configured so that the distal end portion 2242124 of the post is engaged against the head 2138, thereby obstructing the fluid passage 2126 of the post. In some embodiments of the present disclosure, the distal most end of the post 2120 is positioned within the slit 2139 of the head. The head 2138 can include a cavity or concave inner surface that extends from the recess 2136 toward the distal end 2140 of the compressible valve 2114. When the compressible valve 2114 is in the first position, the head 2138 is engaged against the opening of the fluid passage through the post to resist fluid flow therethrough.

When the compressible valve 2114 is in the first position, the head 2138 of the compressible valve can also engage a protrusion 2144 of the housing. The protrusion 2144 extends from the inner surface of the housing 2112 in a direction into the cavity. In some embodiments of the present disclosure, the protrusion 2144 is positioned at the second end 2118 of the housing and extends around the perimeter of the inner surface such that, when the compressible valve 2114 moves toward the first position, for example, when the head 2138 moves toward the second end 2118 of the housing, the head 2138 engages against the protrusion 2144.

In use, when the head 2138 moves toward the second end 2118 of the housing, engagement of the head 2138 against the protrusion 2144 can resist movement of the head 2138 in the direction from the first end 2116 toward the second end 2118 of the housing. Engagement of the head 2138 against the protrusion 2144 can also form a seal between the head 2138 and the housing 2112.

The first valve assembly 2110 is configured to couple with the second valve assembly 2210 by inserting a portion of the second valve assembly 2210 through the second end 2118 of the housing 2112 of the first valve assembly 2110. The first valve assembly 2110 and the second valve assembly 2210 can then be moved toward each other to fluidly couple the first and second valve assemblies 2110, 2210.

FIG. 20 also illustrates a cross-sectional view of the second valve assembly 2210 separated from and spaced apart from the first valve assembly 2110. The second valve assembly 2210 includes a body 2212 having a first end 2216 and a second end 2218. An inner surface of the body 2212 form a bore 2220 that extends through the second end 2218 toward the first end 2216 of the body.

The body 2212 also includes a fluid passage 2226 that extends through the first end 2216 of the body to the bore 2220. The valve plug 2214 is positioned in the bore 2220 and is configured to obstruct the fluid passage 2226 when second valve assembly 2210 is not coupled with the first valve assembly 2110.

The valve plug 2214 includes a first end 2236, a second end 2238, and a slit 2240 that extends through the first and second ends 2236, 2238 of the valve plug. The second end 2238 of the valve plug is aligned with the second end 2218 of the body. In some embodiments of the present disclosure, a common plane intersects the second end 2238 of the valve plug and the second end 2218 of the body.

In some embodiments of the present disclosure, the second end of the body 2212 extends radially inward, in a direction toward the cavity. The portion of the body 2212 extends radially inward can engage against the valve plug 2214 and resist movement of the valve plug 2214 out of the bore 2220.

In some aspects of the present disclosure, the first end 2216 of the body defines a male luer 2242 structure that extends in a direction away from the second end 2218 and forms at least portion of the fluid passage 2226. In some embodiments of the present disclosure, the first end 2116, 2216 of any of the first and second valve assemblies 2110, 2210 can include any of a bond pocket, a female luer, and/or a male luer.

The body 2212 forms a wall 2244 that is configured to engage against a portion of the first valve assembly 2110 when the first and second valve assemblies 2110, 2210 are coupled together. The wall 2244 can be formed by a portion of the body 2212 that extends away from the outer surface in a direction that is transverse, relative to a longitudinal axis A22 extending between the first and second ends 2216, 2218 of the body. In some embodiments of the present disclosure, the wall 2244 can be formed by a portion of the outer surface of the body 2212 that is convex or concave relative to an adjacent portion of the outer surface of the body 2212.

The first and second valve assemblies 2110, 2210 can coupled together by inserting the second end 2218 of the body for the second valve assembly 2210 through the opening into the cavity at the second end 2118 of the housing for first valve assembly 2110, as illustrated in FIG. 13.

When the body 2212 is inserted into the cavity and advanced toward the first end 2116 of the housing, any of the second end 2218 of the body and/or the second end 2238 of the valve plug engages against the distal end 2140 of the compressible valve. As the second valve assembly 2210 is advanced toward the first end 2116 of the housing, the resilient member formed by the proximal end portion 2132 of the compressible valve is compressed and the head moves toward the first end 2116. Movement of the head 2138 toward the first end 2116 of the housing causes the distal end portion 2242124 of the post to move through or pierce the slit 2139 of the head and the slit 2240 of the valve plug.

The body 2212 is advanced toward the first end 2116 of the housing until the protrusion 2144 of the housing moves over and past the wall 2244 of the body, such that the protrusion 2144 is positioned between the wall 2244 and the first end 2216 of the body when the first and second valve assemblies 2110, 2210 are coupled together.

The distance between the wall 2244 and the second end 2218 of the body is configured to resist separation of the first and second valve assemblies 2110, 2210, while maintaining the fluid pathway through the first and second valve assemblies 2110, 2210. The resist separation and maintaining the fluid pathway, the wall 2244 is spaced apart from the second end 2218 of the body is by a distance D21, where the distance D22 is approximately equal to or greater than a sum of the length L21 of the slit 2139 and the length L22 of the slit 2240.

When the first and second valve assemblies 2110, 2210 are coupled together, the opening through the distal end 2127 of the post is positioned between the valve plug 2214 and the first end 2216 of the body so that the fluid passage 2126 of the first valve assembly 2110 is fluidly coupled with the fluid passage 2226 of the second valve assembly 2210.

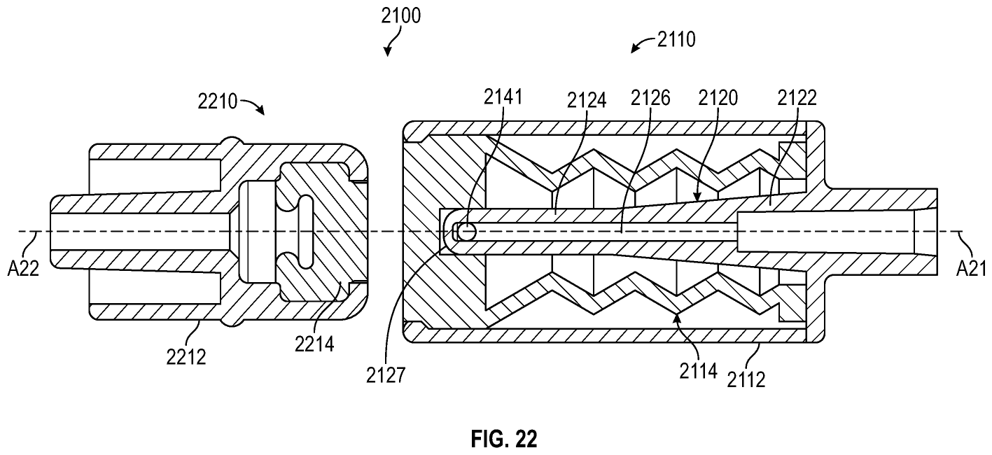

In some embodiments of the present disclosure, such as the embodiment illustrated in FIG. 22, the opening 2141 for the fluid passage is positioned at a location that is spaced apart from the distal end 2127 of the post. The opening 2141 forms a portion of the fluid passage 2126 that extends in a direction that is transverse relative to the longitudinal axis A22 formed by the fluid passage 2126. For example, the opening 2141 at the distal end portion 2242124 of the post can form a portion of the fluid passage 2126 that extend radially outward in a direction that extends away from the longitudinal axis A22.

The opening can be spaced apart from the distal end 2127 of the post and can form a portion of the fluid passage 2126 that extends in a directions that is aligned or parallel to the longitudinal axis A22. In some instances, the distal end 2127 of the post has an outer surface that tapers to form a cross-sectional width that decreases in a direction from the proximal end portion 2122 of the post to the distal end 2127, and the opening extends through the tapered outer surface.

In some embodiments of the present disclosure, the valve plug 2214 can include an inner surface forming a recess 2241 that intersects the slit 2240. When the first and second valve assemblies 2110, 2210 are coupled together, the post of the first valve assembly 2110 can move through the slit 2240 and the recess 2241.

The recess extends into the first end 2236 of the valve plug, in a direction toward the second end 2238 of the valve plug. The recess 2241 can also include a ridge that extends from the inner surface into the recess and in a direction toward the longitudinal axis A22. The ridge can be configured to engage against the post 2242124 of the first valve assembly 2110 to form a seal between the ridge of the valve plug 2214 and the post 2242124. When post 2242124 is positioned through the valve plug 2214, the ridge can resist movement of a fluid between the valve plug 2214 and post 2242124 out of the bore 2220.

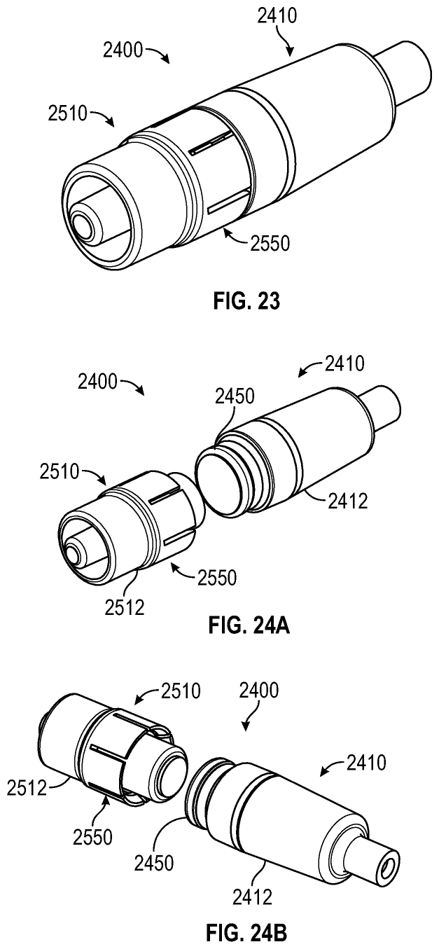

Referring to FIG. 23, an embodiment of a fluid coupling device 2400 is illustrated in which a portion of the first valve assembly 2410 is received into a sleeve 2550 of the second valve assembly 2510 when the first and second valve assemblies 2410, 2510 are coupled together. Features of the fluid coupling device 2400 which are similar to features described with reference to other embodiments herein are not repeated herein for clarity and brevity of the present disclosure.

The fluid coupling device 2400 is illustrated FIGS. 24A and 24B with the first valve assembly 2410 disconnected or separated from the second valve assembly 2510. The first valve assembly 2410 includes a protrusion 2460 that extends in a direction away from an outer surface of the housing 2412. When the first and second valve assemblies 2410, 2510 are coupled together, the protrusion 2460 is inserted into a space between an inner surface of the sleeve 2550 and the outer surface of the body 2212.

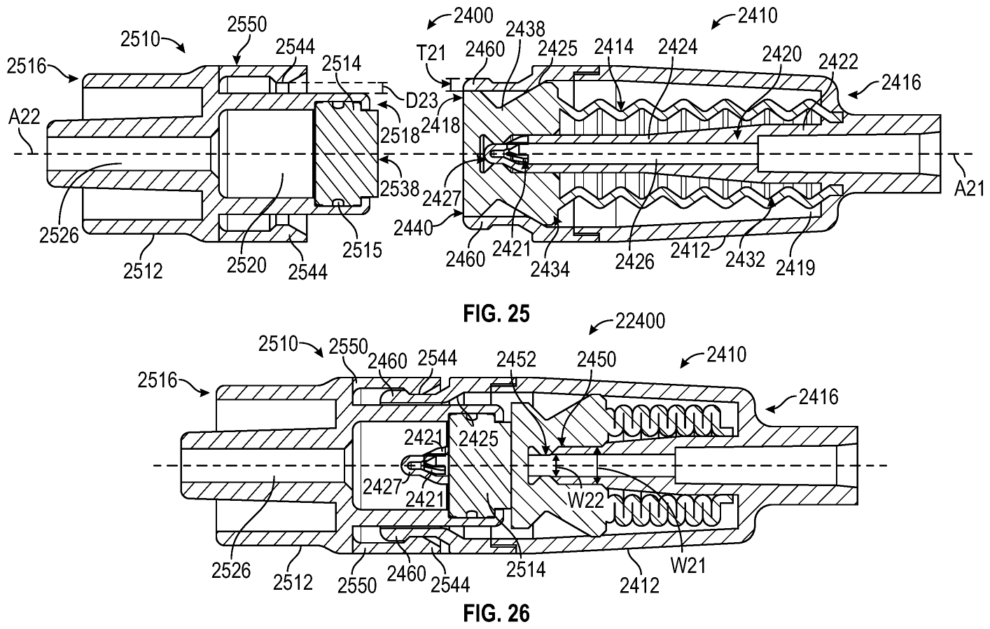

A cross-sectional view of the fluid coupling device 2100 is illustrated in FIGS. 25 and 26. The first valve assembly 2410 includes a housing 2412 having a first end 2416 and a second end 2418. An inner surface of the housing 2412 forms a cavity that extends through the second end 2418 toward the first end 2416 of the housing.

The first valve assembly 2410 also includes a post 2420 that extends within the cavity and forms at least a portion of a fluid passage through the first valve assembly 2410. The post 2420 includes a proximal end portion 2422 and a distal end portion 2424, where the distal end portion 2424 of the post extends within the cavity in a direction from the first end 2416 toward the second end 2418 of the housing.

The post 2420 includes an inner surface forming a fluid passage 2426 that extends through the proximal and distal end portions 2422, 2424 of the post. At the distal end portion 2424 of the post, the fluid passage 2426 extends through an opening 2421 that is spaced apart from a distal end 2427 of the post. In some embodiments of the present disclosure, the opening 2421 extends through a portion of the post having an outer surface that tapers to form a cross-sectional width that decreases in a direction away from the proximal end portion 2422.

A compressible valve 2414 is positioned in the cavity and configured to obstruct the fluid passage 2426 when the compressible valve 2414 is in a first position. The compressible valve 2414 includes a proximal end portion 2432 and a distal end portion 2434. Like the compressible valve 2114 disclosed with reference to FIGS. 20 and 21, the proximal end portion 2432 comprises a resilient member and the distal end portion 2434 comprises a head 2438 having a slit that extends through the head 2438 to a recess within resilient member.

In some embodiments of the present disclosure, the head 2438 comprises an inner surface forming a portion of the recess that extends from the proximal end portion 2432 toward the distal end portion 2434 of the compressible valve. The recess along the head 2438 can be configured so that the head engages against the post to obstruct the opening 2421 when the compressible valve is in the first position.

The portion of the recess along the head 2438 is formed by the inner surface having a cross-section width that is approximately equal to a cross-sectional width of the outer surface of the distal end portion 2424 of the post. In some embodiments of the present disclosure, the portion of the recess along the head 2438 can have a cross-sectional width that tapers to form a decreasing width in the direction from the proximal end portion 2432 toward a distal end 2440 of the head 2438.

To engage against the post, a first portion 2450 of the recess can have a cross-sectional width W1 that is approximately equal to the cross-sectional width of the outer surface of the post located between the proximal end portion 2432 and the opening 2421. A second portion 2452 of the recess, located between the first portion 2450 of the recess and a distal end 2440 of the head, can have a cross-sectional width W2 that is approximately equal to the cross-sectional width of the outer surface of the post located between the opening 2421 and the distal end 2427 of the post.

When the compressible valve 2414 is in a first position, the head 2438 is positioned such that the distal end portion 2424 of the post is within the recess of the head to resist a fluid flow thought the passage 2426 and opening 2421 of the post.

In some embodiments of the present disclosure, when the compressible valve 2414 is in a first position, the inner surface of the head along the first portion 2450 of the recess engages against a portion of the post between the proximal end portion 2432 and the opening 2421, and the inner surface of the head along the second portion 2452 of the recess engages against a portion of the post between the opening 2421 and the distal end 2427 of the post.

When the compressible valve 2414 is in the first position, the head 2438 of the compressible valve can also engage a ledge 2425 of the housing. The ledge 2425 ends from the inner surface of the housing 2412 in a direction into the cavity. In some embodiments of the present disclosure, the ledge 2425 is formed by a portion of the inner surface of the housing having a cross-sectional width that tapers in a direction from the first end 2416 toward the second end 2418 of the housing.

As the compressible valve 2414 moves toward the first position, an outer surface of the head 2438 can engage against the ledge 2425. In some embodiments of the present disclosure, the outer surface of the head 2438 defines a cross-sectional width of the head that tapers so that the cross-sectional width decreases in a direction toward a distal end 2440 of the head.

The housing 2412 also includes a protrusion 2460 that is configured to engage against the second valve assembly 2510 when the first and second valve assemblies 2410, 2510 are coupled together. The protrusion 2460 can be formed by a portion of the housing 2412 that extends away from the outer surface in a direction that is transverse, relative to a longitudinal axis A21 extending between the first and second ends 2416, 2418 of the housing. In some embodiments of the present disclosure, the protrusion 2460 can be formed by a portion of the outer surface of the housing 2412 that is convex or concave relative to an adjacent portion of the outer surface of the housing 2412.

The protrusion 2460 is positioned at the second end 2418 of the housing, and the ledge 2425 is spaced apart from the protrusion 2460 along the longitudinal axis A21. In some aspects of the present disclosure, the distal end 2427 of the post is longitudinally positioned between the ledge 2425 and the protrusion 2460.

The protrusion 2460 is configured to engage against the second valve assembly 2510 to resist unintended separation of the first and second valve assemblies 2410, 2510 from each other. The second valve assembly 2510 can include a body 2512 with an inner bore and a valve plug 2514 positioned in the bore 2520. When the first valve assembly 2410 is not coupled with the second valve assembly 2510, the valve plug 2514 obstructs a fluid passage through the second valve assembly 2510.

In some embodiments of the present disclosure, the valve plug 2514 includes a groove 2515 that extends into an outer surface between the first and second ends of the valve plug. The groove 2515 can extend around the circumference of the valve plug and in a direction toward the longitudinal axis A22 of the second valve assembly 2510. In some aspects, the groove 2515 can permit the valve plug 2514 to flex or bias when the post 2424 is moved through the valve plug 2514.

To resist unintended separation of the first and second valve assemblies 2410, 2510 from each other, the second valve assembly 2510 includes a sleeve 2550. The sleeve extends along the outer surface of the body 2512, in a direction from the first end 2516 toward the second end 2518 of the body. In some embodiments, the sleeve 2550 extends in a direction that is parallel to a longitudinal axis A22 extending between the first and second ends 2516, 2518 of the body.

The sleeve 2550 is spaced apart from the body 2512 to permit insertion of a portion of the first valve assembly 2410 therebetween. The space between the sleeve 2550 and the body 2512 is formed between an inner surface of the sleeve 2550 and an outer surface of the body 2512.

When a portion of the first valve assembly 2410 is positioned between the sleeve 2550 and the body 2512, engagement of the sleeve 2550 and the housing 2412 against each other can resist unintended separation of the first and second valve assemblies 2410, 2510 from each other.

The portion of the sleeve 2550 that engages against the housing 2412 can include a wall 2544. The wall 2544 extends away from the inner surface of the sleeve in a direction toward the outer surface of the body 2512. In some embodiments of the present disclosure, a distal end of the wall is spaced apart from the outer surface of the body 2512 by a distance D23. Where the distance D23 is less that a thickness T21 of the distal end of the housing 2412, the sleeve can flex or bias away from the outer surface of the body 2512 when the second end 2418 of the housing 2412 to be inserted between the sleeve 2550 and the body 2512.

A cross-sectional view of the first and second valve assemblies 2410, 2510 coupled together is illustrated in FIG. 26. When the body 2512 is inserted into the cavity of the housing 2412 and advanced toward the first end 2416 of the housing, any of the second end 2518 of the body and/or the second end 2538 of the valve plug engages against the distal end 2440 of the compressible valve 2414.

As the body 2512 and the housing 2412 are moved toward each other, the second end 2418 of the housing moves into the space between the sleeve 2550 and the outer surface of the body 2512. The body 2512 and the housing 2412 are then moved further toward each other until the protrusion 2460 is positioned between the wall 2544 and the first end 2516 of the body such that engagement of the protrusion 2460 against the wall 2544 resists unintended separation of the first and second valve assemblies 2410, 2510 from each other.

When the body 2512 is inserted into the cavity of the housing 2412, and advanced toward the first end 2416 of the housing, the fluid passage 2426 of the housing becomes fluidly coupled with the fluid passage 2526 of the body when the body 2512 is advanced into the housing 2412 by a distance in which the opening 2421 through the distal end portion of the post is positioned between the valve plug 2514 and the first end 2516 of the body.

In some embodiments of the present disclosure, the distal end 2427 of the post is longitudinally positioned between the ledge 2425 and the protrusion 2460 so that, when the first and second valve assemblies 2410, 2510 coupled together, the opening 2421 through the distal end portion of the post is positioned between the valve plug 2514 and the first end 2516 of the body.

When the first and second valve assemblies 2410, 2510 are coupled together, a fluid pathway of the fluid coupling device 2400 is formed by the fluid passage 2426 of the first valve assembly 2410 and the fluid passage 2526 of the first valve assembly 2510. When the first and second valve assemblies 2410, 2510 are disconnected or separated from each other, the compressible valve 2414 moves to an unrestrained position so that the head 2438 obstructs or resist fluid flow through the post, and the valve plug 2514 resists fluid flow through the fluid passage 2526.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A medical fluid extension set system comprising: a tubing having a first end and a second end; a fluid coupling device coupled to the first end of the tubing; and a tubing anchor coupled to the tubing, between the first and second ends of the tubing.

Clause 2. The medical fluid extension set system of Clause 1, wherein the fluid coupling device comprising a first valve assembly and a second valve assembly, and wherein the first and second valve assemblies are removably coupled together to form a fluid flow path through the tubing and the fluid coupling device.

Clause 3. The medical fluid extension set system of Clause 1, further comprising a catheter coupled to the second end of the tubing.

Clause 4. The medical fluid extension set system of Clause 1, wherein a length of the tubing, between the anchor and the second end of the tubing, is greater that a distance between the anchor and the second end of the tubing.

Clause 5. The medical fluid extension set system of Clause 1, wherein the tubing anchor is coupled to the tubing such that movement of the tubing anchor along a length of the tubing between the first and second ends of the tubing is resisted.

Clause 6. The medical fluid extension set system of Clause 1, wherein a portion of the tubing, between the anchor and the second end of the tubing, comprises a non-linear shape when the portion of the tubing is not biased.

Clause 7. The medical fluid extension set system of Clause 1, wherein a portion of the tubing, between the anchor and the second end of the tubing, comprises a bellows.

Clause 8. The medical fluid extension set system of Clause 1, further comprising an adhesive layer that extends along an outer surface of the tubing anchor.

Clause 9. The medical fluid extension set system of Clause 8, wherein the tubing anchor comprises a top surface defining a first surface area, and the adhesive layer defines a second surface area, and wherein the second surface area is greater than the first surface area.

Clause 10. The medical fluid extension set system of Clause 1, wherein the tubing anchor comprises an anchor body having a first end, a second end, a first sidewall, a second sidewall, a top end, a bottom end, and a tubing passage extending through the first and second ends and defining a longitudinal axis of the anchor body.

Clause 11. The medical fluid extension set system of Clause 10, wherein the tubing anchor comprises a width between the first and second sidewalls, and wherein the width of the tubing anchor decreases in a direction from the first end of the tubing anchor toward the second end of the tubing anchor.

Clause 12. The medical fluid extension set system of Clause 11, wherein the tubing extends through the tubing anchor such that the second end of the tubing anchor is oriented toward the fluid coupling device.

Clause 13. The medical fluid extension set system of Clause 10, wherein the first sidewall comprises a front portion and a rear portion, and the front and rear portions of the first sidewall extend away from the longitudinal axis and intersect at an apex of the first sidewall, and wherein the second sidewall comprises a front portion and a rear portion, and the front and rear portions of the second sidewall extend away from the longitudinal axis and intersect at an apex of the second sidewall.

Clause 14. The medical fluid extension set system of Clause 13, wherein a width of the tubing anchor between the apex of the first and second sidewalls, is greater than a height of the tubing anchor between the top and bottom ends.

Clause 15. The medical fluid extension set system of Clause 13, wherein a first length of the tubing anchor, extending from the apex of the first sidewall to the apex of the second sidewall, and along the second end of the tubing anchor, is greater than a second length of the tubing anchor, extending from the apex of the first sidewall to the apex of the second sidewall, and along the first end of the tubing anchor.

Clause 15. The medical fluid extension set system of Clause 1, wherein a fluid connector is coupled to the second end of the tubing, and a portion of the fluid connector is coupled to the tubing anchor.

Clause 16. The medical fluid extension set system of Clause 15, wherein the fluid connector is movable relative to the tubing anchor.

Clause 17. The medical fluid extension set system of Clause 1, further comprising a fluid connector coupled between the first end of the tubing and the fluid coupling device.

Clause 18. The medical fluid extension set system of Clause 1, wherein the fluid coupling device is pivotably coupled to the tubing anchor.

Clause 19. The medical fluid extension set system of Clause 1, further comprising another tubing having a first end coupled to the fluid coupling device, and another tubing anchor coupled to the another tubing, wherein the fluid coupling device is fluidly coupled between the tubing anchor and the another tubing anchor.

Clause 20. The medical fluid extension set system of Clause 1, wherein the fluid coupling device comprises: a first valve assembly comprising: a housing having a first end, a second end, wherein the second end is opposite to the first end, and an inner surface forming a cavity that extends through the second end toward the first end of the housing; a post having a proximal end portion and a distal end portion, wherein the distal end portion of the post extends within the cavity in a direction from the first end of the housing toward the second end of the housing; a fluid passage that extends through the first end of the housing and the post; and a compressible valve positioned in the cavity, the compressible valve having a proximal end portion and a distal end portion, wherein the proximal end portion comprises a resilient member having an inner surface forming a recess, and wherein the distal end portion comprises a head having a slit that extends through the head to the recess; a second valve assembly comprising: a body having a first end, a second end, wherein the second end is opposite to the first end, an inner surface forming a bore that extends through the second end toward the first end of the body, and a fluid passage that extends through the first end of the body to the bore; a valve plug positioned in the bore, the valve plug having a first end, a second end, and a slit that extends through the first and second ends of the valve plug.

Clause 21. The medical fluid extension set system of Clause 20, wherein, when the first and second valve assemblies are separated from each other, the compressible valve is in a first position with a distal end of the head aligned with the second end of the housing and the distal end portion of the post positioned within the recess, and when the first and second valve assemblies are coupled together, the second end of the body is positioned within the cavity of the housing such that the compressible valve is in a second position with the head biased toward the first end of the housing and the distal end portion of the post extending through the slit of the head of the compressible valve and through the slit of the valve plug, such that the fluid passage of the first valve assembly is fluidly coupled with the fluid passage of the second valve assembly.

Clause 22. A method of providing a medical fluid extension set system, the method comprising: providing a tubing having a first end and a second end, wherein the first end comprises a fluid coupling device; and coupling a tubing anchor to the tubing, such that the tubing anchor is positioned between the first and second ends of the tubing.

Clause 23. The method of Clause 22, wherein coupling the tubing anchor to the tubing comprises inserting the tubing through a tubing passage that extends through a first end of the tubing anchor and a second end of the tubing anchor

Clause 24. The method of Clause 22, further comprising bonding the tubing anchor to the tubing such that movement of the tubing anchor along a length of the tubing between the first and second ends of the tubing is resisted.

Clause 25. The method of Clause 22, further comprising coupling a catheter to the second end of the tubing.

Clause 26. The method of Clause 22, wherein a length of the tubing, between the anchor and the second end of the tubing, comprises a non-linear shape, such that a length of the tubing between the anchor and the second end of the tubing is greater that a distance between the anchor and the second end of the tubing.

Clause 27. The method of Clause 22, further comprising providing an adhesive layer configured to engage against an outer surface of the tubing anchor and a patient.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A medical fluid extension set system comprising:
a tubing having a first end and a second end;
a fluid coupling device coupled to the first end of the tubing; and
a tubing anchor coupled to the tubing, between the first and second ends of the tubing.

2. The medical fluid extension set system of claim 1, wherein the fluid coupling device comprising a first valve assembly and a second valve assembly, and wherein the first and second valve assemblies are removably coupled together to form a fluid flow path through the tubing and the fluid coupling device.

3. The medical fluid extension set system of claim 1, further comprising a catheter coupled to the second end of the tubing.

4. The medical fluid extension set system of claim 1, wherein the tubing anchor is coupled to the tubing such that movement of the tubing anchor along a length of the tubing between the first and second ends of the tubing is resisted.

5. The medical fluid extension set system of claim 1, further comprising an adhesive layer that extends along an outer surface of the tubing anchor.

6. The medical fluid extension set system of claim 1, wherein the tubing anchor comprises an anchor body having a first end, a second end, a first sidewall, a second sidewall, a top end, a bottom end, and a tubing passage extending through the first and second ends and defining a longitudinal axis of the anchor body.

7. The medical fluid extension set system of claim 6, wherein the tubing anchor comprises a width between the first and second sidewalls, and wherein the width of the tubing anchor decreases in a direction from the first end of the tubing anchor toward the second end of the tubing anchor.

8. The medical fluid extension set system of claim 7, wherein the tubing extends through the tubing anchor such that the second end of the tubing anchor is oriented toward the fluid coupling device.

9. The medical fluid extension set system of claim 6, wherein the first sidewall comprises a front portion and a rear portion, and the front and rear portions of the first sidewall extend away from the longitudinal axis and intersect at an apex of the first sidewall, and wherein the second sidewall comprises a front portion and a rear portion, and the front and rear portions of the second sidewall extend away from the longitudinal axis and intersect at an apex of the second sidewall.

10. The medical fluid extension set system of claim 9, wherein a width of the tubing anchor between the apex of the first and second sidewalls, is greater than a height of the tubing anchor between the top and bottom ends.

11. The medical fluid extension set system of claim 9, wherein a first length of the tubing anchor, extending from the apex of the first sidewall to the apex of the second sidewall, and along the second end of the tubing anchor, is greater than a second length of the tubing anchor, extending from the apex of the first sidewall to the apex of the second sidewall, and along the first end of the tubing anchor.

12. The medical fluid extension set system of claim 1, wherein a fluid connector is coupled to the second end of the tubing, and a portion of the fluid connector is coupled to the tubing anchor.

13. The medical fluid extension set system of claim 1, wherein the fluid coupling device is pivotably coupled to the tubing anchor.

14. The medical fluid extension set system of claim 1, further comprising another tubing having a first end coupled to the fluid coupling device, and another tubing anchor coupled to the another tubing, wherein the fluid coupling device is fluidly coupled between the tubing anchor and the another tubing anchor.

15. The medical fluid extension set system of claim 1, wherein the fluid coupling device comprises:
a first valve assembly comprising:
a housing having a first end, a second end, wherein the second end is opposite to the first end, and an inner surface forming a cavity that extends through the second end toward the first end of the housing;
a post having a proximal end portion and a distal end portion, wherein the distal end portion of the post extends within the cavity in a direction from the first end of the housing toward the second end of the housing;
a fluid passage that extends through the first end of the housing and the post; and
a compressible valve positioned in the cavity, the compressible valve having a proximal end portion and a distal end portion, wherein the proximal end portion comprises a resilient member having an inner surface forming a recess, and wherein the distal end portion comprises a head having a slit that extends through the head to the recess;
a second valve assembly comprising:
a body having a first end, a second end, wherein the second end is opposite to the first end, an inner surface forming a bore that extends through the second end toward the first end of the body, and a fluid passage that extends through the first end of the body to the bore; and
a valve plug positioned in the bore, the valve plug having a first end, a second end, and a slit that extends through the first and second ends of the valve plug.

16. The medical fluid extension set system of claim 15, wherein, when the first and second valve assemblies are separated from each other, the compressible valve is in a first position with a distal end of the head aligned with the second end of the housing and the distal end portion of the post positioned within the recess, and when the first and second valve assemblies are coupled together, the second end of the body is positioned within the cavity of the housing such that the compressible valve is in a second position with the head biased toward the first end of the housing and the distal end portion of the post extending through the slit of the head of the compressible valve and through the slit of the valve plug, such that the fluid passage of the first valve assembly is fluidly coupled with the fluid passage of the second valve assembly.

17. A method of providing a medical fluid extension set system, the method comprising:
providing a tubing having a first end and a second end, wherein the first end comprises a fluid coupling device; and
coupling a tubing anchor to the tubing, such that the tubing anchor is positioned between the first and second ends of the tubing.

18. The method of claim 17, wherein coupling the tubing anchor to the tubing comprises inserting the tubing through a tubing passage that extends through a first end of the tubing anchor and a second end of the tubing anchor.

19. The method of claim 17, further comprising bonding the tubing anchor to the tubing such that movement of the tubing anchor along a length of the tubing between the first and second ends of the tubing is resisted.

20. The method of claim 17, further comprising coupling a catheter to the second end of the tubing.
